(19) United States
(12) Patent Application Publication
(10) Pub. No.: US 2013/0018408 A1
(43) Pub. Date: Jan. 17, 2013

(54) Minimally Invasive Dilator and Retractor System

(71) Applicant: Daniel K. Farley, Traverse City, MI (US)

(72) Inventor: Daniel K. Farley, Traverse City, MI (US)

(21) Appl. No.: 13/183,085

(22) Filed: Jul. 14, 2011

(51) Int. Cl. A61M 29/00

(52) U.S. Cl. 606/198

(57) ABSTRACT

A novel dilation system for providing access to a surgical site of interest is provided. The dilator includes a body, an arm, and a plurality of articulating dilation members pivotally connected to the body. The body includes a cavity, and the arm is configured to be accepted by the cavity of the body. The arm includes a distal portion and a proximal portion. The plurality of articulating dilation members are operably connected to the distal portion of the arm, and are movable between a closed position and an open position. The dilator may define a distally tapering generally conical volume in the closed position, and a generally cylindrical volume in the open position. The articulating dilation members are moved from the closed position toward the open position by the arm when the arm is articulated distally, and moved from the open position to the closed position by the arm when the arm is articulated proximally. The arm and the plurality of articulating dilation members include cooperating surfaces that maintain the articulating dilation members in the closed position when the arm is held in place during insertion of the dilator.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The disclosed inventions relate to dilation systems. In particular, the inventions relate to dilation systems useful for minimally invasive surgical procedures.

In the past, surgery typically required large incisions to provide access for visualization and instrument placement and manipulation relative to the surgical site. These large incisions could result in significant blood loss, damage to muscle tissue, increased healing times accompanied by prolonged pain, and significant scarring. Currently, many surgeries are conducted using minimally invasive techniques. These techniques minimize patient trauma by creating a relatively small incision, followed by the introduction of dilators to increase the effective size of the incision. Following dilation, surgery is performed through a surgical port inserted into the dilated incision. Instead of cutting through the muscle surrounding the surgical site, dilation effectively splits the muscle. Splitting, rather than cutting the muscle causes less damage to the muscle and leads to faster recovery times and reduced patient discomfort.

Certain known dilators develop a channel from the subcutaneous layer of a patient to the site of operation. In certain procedures, a small incision, paralleling any underlying muscle, is made slightly longer than ½ the circumference of the largest dilator, or if used, port. A solid, pointed rod, variously described as a first dilator or guidewire, is then inserted into the incision to penetrate the underlying structures and reach the surgical site. It is best if the rod can be positioned against a bony surface as application of the dilators will attempt to push this rod forward. X-rays may be taken before and/or after insertion of the rod to confirm placement at the desired surgical site.

Increasingly larger diameter dilators can then be sequentially placed over each other to enlarge the channel. The larger diameters of the sequential dilators help to dilate the path of exposure while the series of tubes lessens the forces needed to create the path. The pointed tip of the dilators eases insertion and helps to widen the base of the channel when the dilator is orbited around a central axis formed through the center of the dilator along its length at the level of the skin.

In lieu of dilation, mechanical retractors can be used. Mechanical surgical retractors are hand-held or table-mounted metal retractor blades that are inserted into the incision, and thereafter retracted and held or locked in place to increase the effective opening of the incision. A drawback of using certain known retractors is that, in comparison to dilators, a relatively large incision must be made to provide for placement of the retractor blades.

Conventional dilators and surgical ports, however, are not suitable for all surgical applications. For example, conventional dilators are unable to completely dilate muscle away from the lamina of the spine due to the tortuous geometry of the lamina. Thus, muscle located between the dilator and the lamina must typically be cut away to access the lamina when using conventional dilators.

As another example, U.S. Pat. No. 7,407,483 (Perez-Cruet et al., “Minimally Invasive Surgical Access Device”), which is hereby incorporated by reference in its entirety, discloses a device that has retractor blades that are threaded into a patient with the assistance of an insertion handle. Then, the insertion handle is removed, and a core hollow screw is threaded into the base portion to separate the retractor blades to gain access to the pathology through the hollow screw.

Due to the geometry of the spine, many spinal surgical procedures require a long, narrow opening. Thus, another drawback of dilators or ports is that a circular opening may not be practical for certain spinal surgeries because of the limited access it offers to the spine given the size of the dilated opening. The use of dilators and surgical ports may therefore be limited to procedures involving very precise access to the spine, such as for single level discectomy.

Mechanical retractors, on the other hand, may provide a long, narrow opening. As discussed above, however, mechanical retractors may require a relatively large initial incision that involves cutting, rather than splitting of muscle.

### BRIEF SUMMARY OF THE INVENTION

In certain embodiments of the present invention, a novel dilation system for providing access to a surgical site of interest is provided. The dilator includes a body, an arm, and a plurality of articulating dilation members pivotally connected to the body. The body includes a cavity, and the arm is configured to be accepted by the cavity of the body. The arm includes a distal portion and a proximal portion. The plurality of articulating dilation members are operably connected to the distal portion of the arm, and are movable between a closed position and an open position. The dilator may define a distally tapering generally conical volume in the closed position, and a generally cylindrical volume in the open position. The articulating dilation members are moved from the closed position toward the open position by the arm when the arm is articulated distally, and moved from the open position to the closed position by the arm when the arm is articulated proximally. The arm and the plurality of articulating dilation members include cooperating surfaces that maintain the articulating dilation members in the closed position when the arm is held in place during insertion of the dilator.

Further, in certain embodiments, the cavity of the body includes a threaded section and the arm includes an intermediate portion including a threaded portion. The threaded portion of the body accepts the threaded portion of the arm, wherein the arm may be articulated linearly relative to the body when the arm is rotated. Further, the distal portion of the arm may include a conically shaped portion including a sloped surface having a distally reduced cross sectional area, wherein the sloped surface actuates the articulating dilation members toward the open position when the arm is articulated distally. Further still, the distal portion of the arm may include a rear surface located proximally of the sloped surface. Each articulating dilation member may include an ear, wherein the rear surface of the arm contacts the ears and moves the articulating dilation members from the open position to the closed position when the arm is articulated proximally. In certain embodiments, the rear surface of the arm may contact the ears and prevent the articulating dilation members from pivoting toward the open position when the articulating dilation members are in the closed position. In certain embodiments, the ear comprises a protrusion. The protrusion contacts a first surface of the arm to prevent opening, and contacts a second surface of the arm to limit opening.

In certain embodiments, the articulating dilation members comprise external surfaces having threads. The articulating dilation members define a substantially continuous threaded surface when the articulating dilation members are in the closed position. Further still, the cavity of the body includes a threaded section and the arm includes an intermediate portion including a threaded portion. The threaded portion of the body accepts the threaded portion of the arm, wherein the arm may be articulated linearly relative to the body when the arm is rotated. Rotation in a first direction advances the arm distally relative to the body, and rotation in a second, opposite direction advances the articulating dilation members through tissue.

Certain embodiments of the present invention provide a novel dilation and retraction system for providing access to a surgical site of interest. The dilator includes a body, an arm, and a plurality of articulating dilation members pivotally connected to the body. The body includes a cavity, and the arm is configured to be accepted by the cavity of the body. The arm includes a distal portion and a proximal portion. The plurality of articulating dilation members are operably connected to the distal portion of the arm, and are movable between a closed position and an open position. The articulating dilation members are moved from the closed position toward the open position by the arm when the arm is articulated distally, and moved from the open position to the closed position by the arm when the arm is articulated proximally. The arm and the plurality of articulating dilation members include cooperating surfaces that maintain the articulating dilation members in the closed position when the arm is held in place during insertion of the dilator. The retractor is configured to be slid over the dilator when the dilator is in the open position. For example, the retractor may comprise a plurality of retractor blades that are configured to be slid over the dilator in a closed retractor position toward the surgical site of interest, and then moved to an open retractor position. The dilator may define a generally cylindrical envelope in the open position, and the retractor may define a generally circular cross-section configured to be slid over the dilator.

Certain embodiments of the present invention provide a method for providing access to a surgical site of interest. The method includes making an incision. The method also includes advancing a dilator having articulating dilation members in a closed position into a patient toward a site of interest. The dilator has a generally conical distal end when in the closed position. The method further includes moving the articulating dilation members to an open position, with the distal end of the dilator defining a generally cylindrical volume in the open position. Also, the method includes inserting a retractor over the dilator toward the site of interest, positioning the retractor as desired to provide the desired access to the site of interest, and removing the dilator to provide access to the site of interest. In certain embodiments, the dilator includes a threaded arm that spreads the articulating dilation members when the threaded arm is advance distally, and the dilator includes a threaded exterior. Advancing the dilator further includes rotating the dilator in a first direction, and moving the articulating dilation members to the open position includes rotating the threaded arm in a second direction opposite to the first direction.

### BRIEF DESCRIPTION OF SEVERAL VIEWS OF THE DRAWINGS

FIG. 1 illustrates a perspective view of a dilator formed in accordance with an embodiment of the present invention in a closed position.

FIG. 2 illustrates a perspective view of the dilator of FIG. 1 in an open position.

FIG. 3 illustrates a sectional view of the dilator of FIG. 1 in the closed position.

FIG. 4 illustrates a sectional view of the dilator of FIG. 1 in the open position.

FIG. 5 illustrates a side view of a drive arm body conned in accordance with an embodiment of the present invention.

FIG. 6 illustrates a perspective view of a drive arm extension formed in accordance with an embodiment of the present invention.

FIG. 7 illustrates a perspective view of a body for a dilator formed in accordance with an embodiment of the present invention.

FIG. 8 illustrates a top perspective view of a blade formed in accordance with an embodiment of the present invention.

FIG. 9 illustrates a bottom perspective view of the blade of FIG. 8.

FIG. 10 illustrates a perspective view of a dilation and retraction system formed in accordance with an embodiment of the present invention.

### DETAILED DESCRIPTION OF THE INVENTION

Reference now will be made in detail to certain embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For example, features illustrated or described as part of one embodiment, may be used on or with another embodiment to yield a still further embodiment. Thus, it is intended that the present invention cover such modifications and variations. Other objects, features and aspects of the present invention are disclosed in or are apparent from the following detailed description. It is to be understood by one of ordinary skill in the art that the present discussion is a description of exemplary embodiments only, and is not intended as limiting the broader aspects of the present invention.

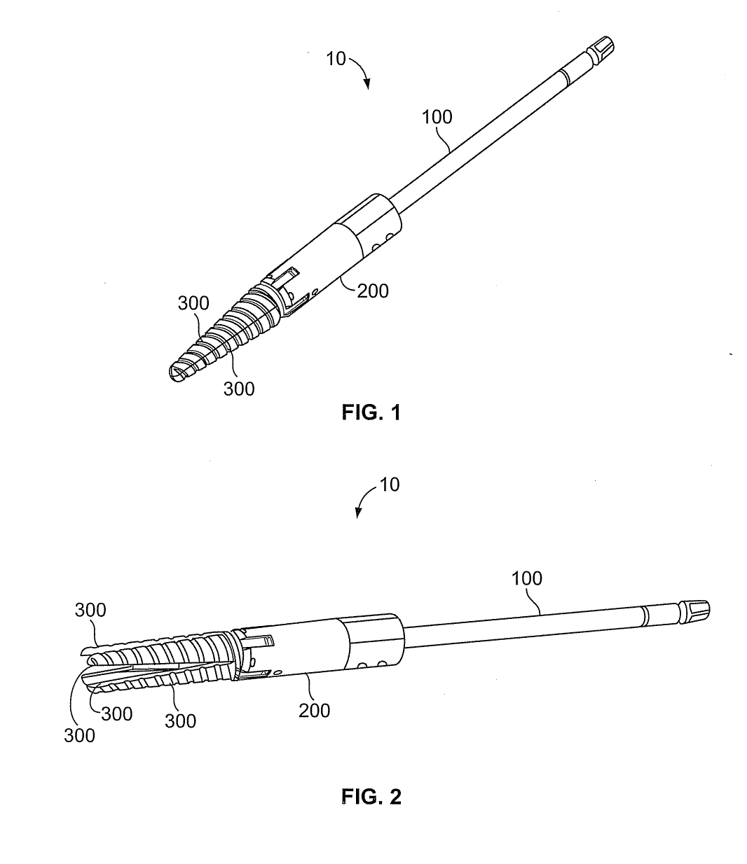

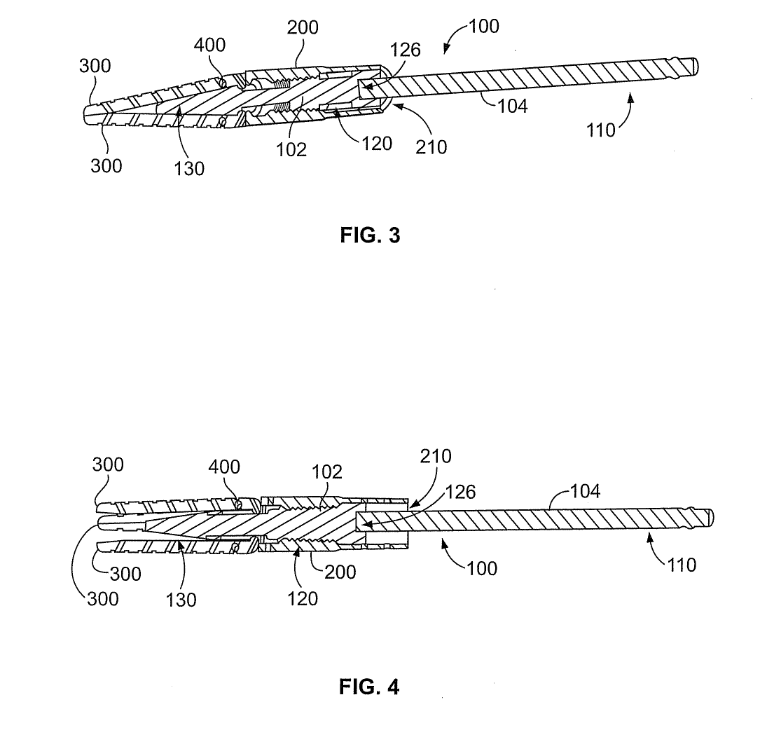

FIGS. 1 and 2 illustrate a perspective view of a dilator 10 formed in accordance with an embodiment of the present invention. The dilator 10 may be used, for example, for posterior lumbar procedures. In FIG. 1, the dilator 10 is shown in a closed position, and in FIG. 2 the dilator 10 is shown in an open position. Also, FIG. 3 illustrates a sectional view of the dilator 10 in the closed position, and FIG. 4 illustrates a sectional view of the dilator 10 in the open position. The dilator 10 is configured to be inserted into an incision toward a site of interest. Once the desired position is achieved, the dilator 10 is expanded, and a retractor assembly and/or individual retractors may be slid over the dilator 10 and positioned. Once the retractor blades are positioned, the dilator 10 may be removed, providing a practitioner with access to the site of interest. Examples of retractor assemblies and/or individual retractors that may be used with dilators formed in accordance with certain embodiments of the present invention may be found for, example, in U.S. patent application Ser. No. 12/792,313 (“Malleable Port Retractor,” filed Jun. 2, 2010), U.S. patent application Ser. No. 12/959,701 (“Posterior Lumbar Retractor System,” filed Dec. 3, 2010) and U.S. patent application Ser. No. 13/087,866 (“Lateral and Anterior Lateral Retractor System,” filed Apr. 15, 2011), each of which is incorporated herein by reference in its entirety.

The dilator 10 includes a drive arm 100, a body 200, and a plurality of blades 300. In the illustrated embodiment, the dilator 10 includes four blades 300. The blades 300 are an example of articulating dilation members. In alternate embodiments, fewer or more articulating dilation members could be employed. The dilator 10 is configured so that the arm 100 spreads the blades 300 from a first, or closed, position, toward a second, or open, position, when the arm 100 is advanced distally through the body 200. Similarly, the dilator 10 is configured so that the arm 100 returns the blades 300 from the second, or open, position, toward the first, or closed, position, when the arm 100 is articulated proximally through the body 200. Different configurations may be used in alternated embodiments. For example, in certain embodiments, a proximal movement of the arm may open the blades while a distal movement of the arm closes the blades.

The dilator 10 has a number of advantages in use. The dilator 10 is inserted in one piece, so minimizes the use, addition, and removal of additional pieces. Its tapered design and opening blades allows for efficient insertion and dilation, and helps minimize tissue damage during insertion and dilation. The use of retractors placed over the top of the dilator and then expanded to a desired position, followed by removal of the dilator allows for a larger access area with more flexibility than if the envelope created by the dilator alone were used for access.

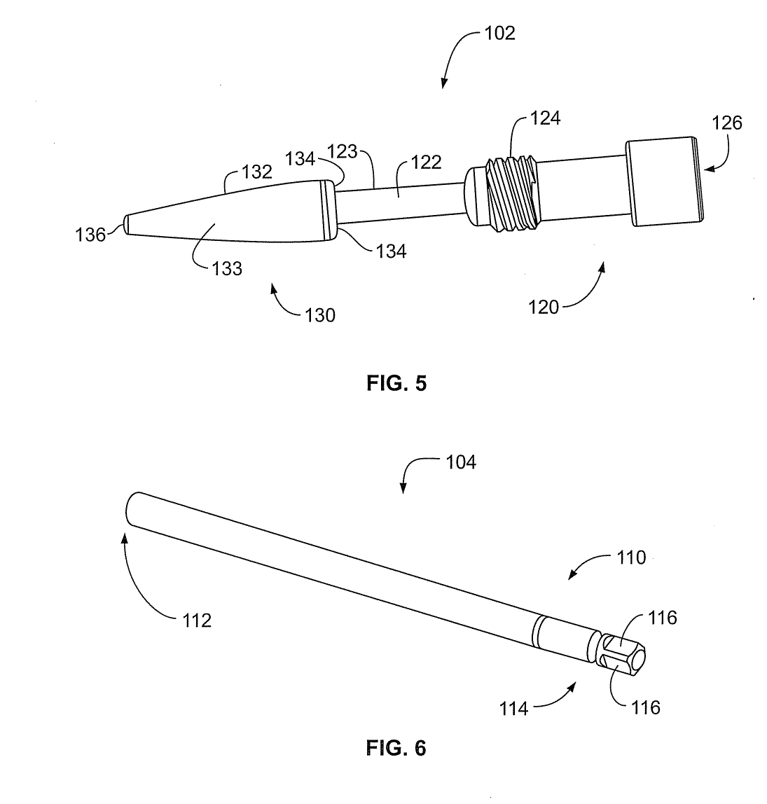

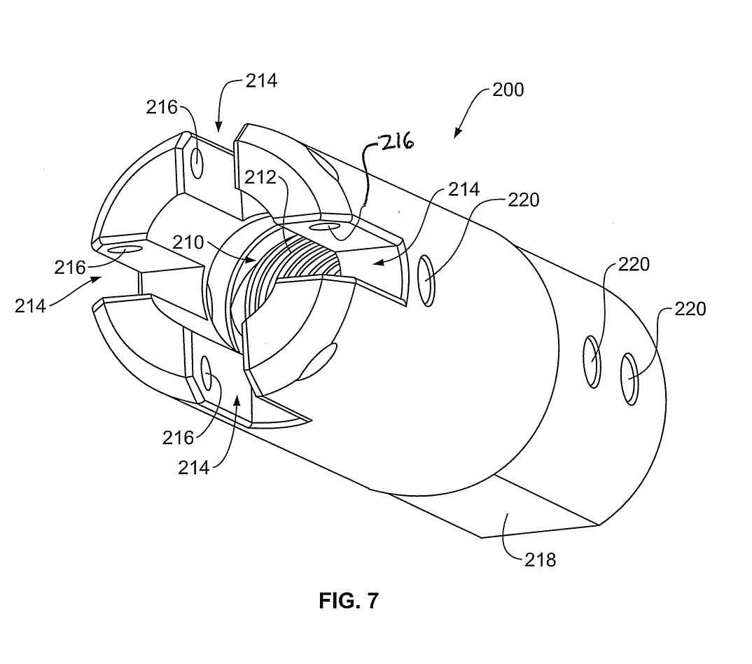

The drive arm 100 of the illustrated embodiment includes a drive arm body 102 and a drive arm extension 104. FIG. 5 illustrates a side view of a drive arm body 102 formed in accordance with an embodiment of the present invention, and FIG. 6 illustrates a perspective view of a drive arm extension 104 formed in accordance with an embodiment of the present invention. The drive arm body 102 is configured to accept the drive arm extension 104. The drive arm body 102 is accepted by the body 200 and interacts with the blades 300 to open and close the blades, depending on which direction the drive arm body 200 is actuated. The drive arm extension 104 provides a convenient surface for grasping and manipulating, and allows a practitioner to actuate the drive arm body 102. For example, with the drive arm extension 104 mounted in the drive arm body 102, a practitioner may turn the drive arm extension 104, which will in turn cause the drive arm body 102 to rotate. In the illustrated embodiment, the drive arm 100 includes a proximal portion 110, an intermediate portion 120, and a distal portion 130. The drive arm extension 104 includes the proximal portion 110, while the drive arm body 102 includes the intermediate portion 120 and the distal portion 130. In alternate embodiments, the drive arm 100 could be made from a single piece, or, in still other embodiments, the drive arm 100 could be made from more than two pieces.

In the illustrated embodiment, the drive arm body 104 includes a distal portion 130 and an intermediate portion 120. The drive arm body 104 includes a shaft 122, threaded portion 124, and a receiving cavity 126. The threaded portion 124 is interposed between the shaft 122 and the receiving cavity 126. The threaded portion 124 is configured to be accepted in threaded engagement with an internal thread of the body 200. Thus, by rotating the drive arm 100 in one direction respective to the body 200, the drive arm 100 may be actuated distally through the body 200, while rotating in the opposite direction results in the drive arm 100 being actuated proximally through the body 200. The shaft 122 joins the threaded portion 124 to the distal portion 130. In the illustrated embodiment, the shaft 122 is a generally cylindrical shape and is of a reduced diameter relative to the largest diameter of the distal portion 130 as well as the threaded portion 124. The shaft 122 includes an exterior contact surface 123 (discussed below also).

The distal portion 130 of the drive arm 100 of the illustrated embodiment is a generally conically shaped member configured to open the blades 300 when the drive arm 100 is actuated distally, and to close the blades 300 when the drive arm is actuated proximally. The distal portion 130, which provides an example of a blade actuating portion, includes a wedge portion 132 interposed between a tip 136 and a rear surface 134. The rear surface 134 extends generally perpendicularly from the distal end of the shaft 122. The transition between the shaft 122 and the rear surface 134, as well as the transition between the rear surface 134 and the wedge portion 132, may be radiused.

The wedge portion 132 is shaped as a generally conical section, including a sloped exterior section 133. The sloped exterior section 133 is configured to interact with interior, or bottom, surfaces of the blades. The tip 136 is generally parallel to the rear surface 134, and is configured to help provide for a clearance between the distal end of the distal portion 134 and the point where the blades 300 join each other when the blades 300 are closed.

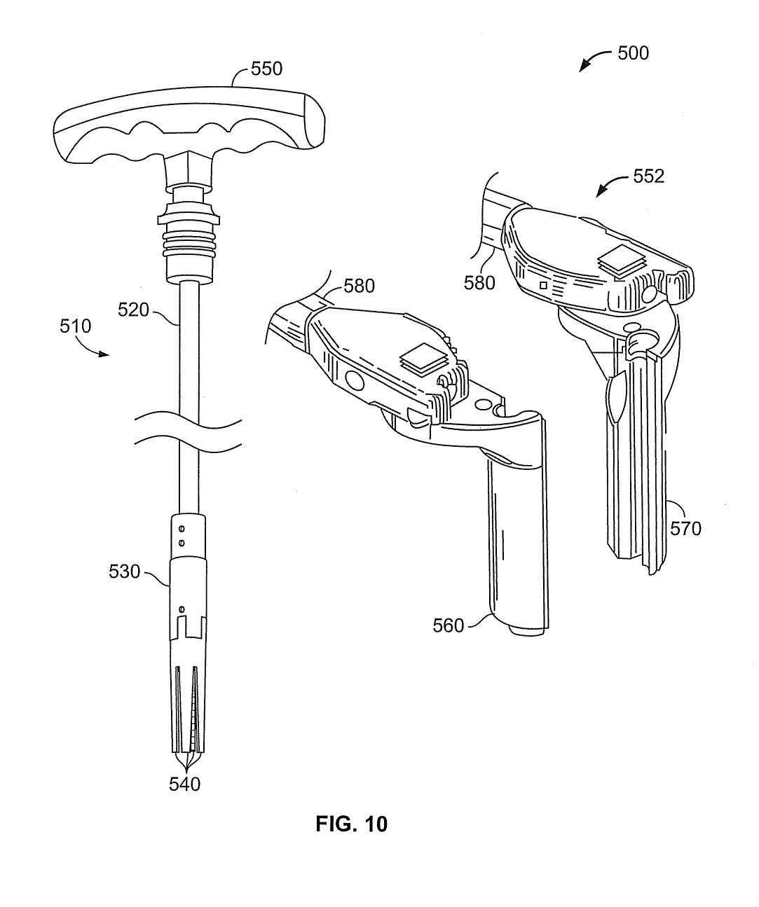

As seen in FIG. 6, the drive arm extension 104 is generally cylindrical and includes an insertion end 112 and a manipulation end 114. The insertion end 112 is configured to be accepted by and/or mounted to the receiving cavity 126 of the drive arm body 102 such that the drive arm body 102 rotates when the drive arm extension 104 is rotated. For example, the insertion end 112 may be welded in the receiving cavity 126. In alternate embodiments, for example, the drive arm body 102 and drive arm extension 104 may be machined from a single piece. The manipulation end 114 is configured to be grasped to allow for convenient rotation of the drive arm extension 104 (and, consequently, rotation of the drive arm body 102 when the drive arm extension 104 is accepted by the drive arm body 102). For example, in the illustrated embodiment, the manipulation end 114 includes flats 116. These flats 116 may be grasped by a wrench for convenient rotation of the drive arm 100. Alternatively, the flats 116 (or other surface) could be accepted by a cavity of a handle (not shown in FIGS. 1-6; for example of a handle see FIG. 10) that a practitioner may grasp and turn. In still further alternate embodiments, flats, protrusions, or other surfaces may be provided elsewhere along the length of the drive arm extension 104.

FIG. 7 illustrates a perspective view of a body 200 formed in accordance with an embodiment of the present invention. The body 200 is configured to pivotally connect with the blades 300, and to accept the drive arm 100 such that actuation of the drive arm pivots the blades 300 about their connection with the body 200. In the illustrated embodiment, the drive arm 100 is threadedly engaged with the body 200, such that a counter-clockwise rotation of the drive arm 100 results in a distal actuation of the drive arm 100 to open the blades 300, and a clockwise rotation of the drive arm 100 results in a proximal actuation of the drive arm 100 to close the blades 300.

The body 200 is generally cylindrically shaped and includes a cavity 210 (see also FIGS. 3 and 4), slots 214, pin holes 216, flats 218 located on opposing sides of the body 200 proximate to the proximal end of the body 200, and drain holes 220.

The cavity 210 includes a threaded portion 212. The threaded portion 212 accepts the threaded portion 124 of the drive arm body 104 in threaded engagement. For example, a counter-clockwise rotation of the drive arm body 104 may advance the drive arm body 104 distally relative to the body 200, and a clockwise rotation actuates the drive arm body 104 proximally relative to the body 200.

The slots 214 are formed toward the distal end of the body 200 and are configured to accept, with clearance, ears or tabs of the blades 300. The slots 214 are sized to allow the ears or tabs of the blades 300 to rotate in the slots 214. In the illustrated embodiment, each blade 300 has one ear or tab, and the dilator 10 has four blades 300, so the body has four slots 214. In alternate embodiments, different numbers and/or configurations of slots may be employed. Pin holes 216 extend generally perpendicularly on each side of each slot 214. The pin holes 216 are configured to accept pins 400 (see FIGS. 3 and 4). The pins pivotally connect the blades 300 to the body 200.

The body 200 also includes flats 218 located toward the proximal end of the body 200. The flats 218 are configured to provide a convenient surface for grasping the body 200, for example with a wrench, or, as another example, with a clamp secured by a frame to an operating table, to assist in rotating the body 200 and/or maintaining the body 200 in a stationary position while the drive arm 100 is being rotated relative to the body. The body 200 also includes drain holes 220.

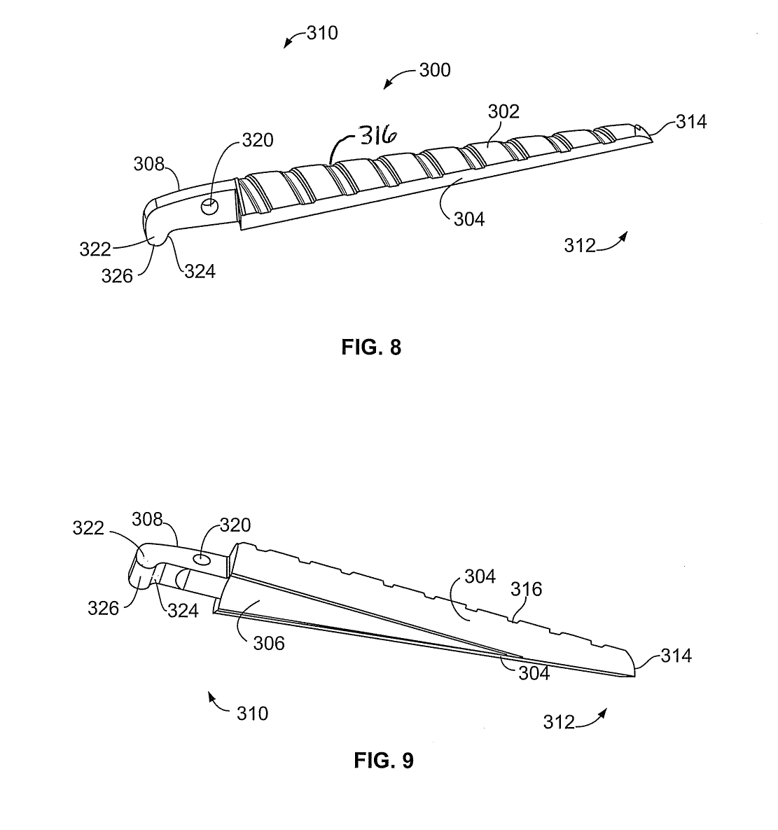

FIG. 8 illustrates a top perspective view of a blade 300 formed in accordance with an embodiment of the present invention, and FIG. 9 illustrates a bottom perspective view of the blade 300. The blade includes a top surface 302, side surfaces 304, a bottom surface 306, and an ear 308. In the illustrated embodiment, the dilator 10 includes four blades 300, but fewer or more blades could be used in alternate embodiments. The blades, for example, may be made of aluminum or titanium, which allows them to be radiolucent.

The top surface 302 is formed to be a portion of a generally conical segment. When the four blades 300 are in place in the closed position, they cooperate to form a generally conical member, with the top surfaces 302 of the blades 300 cooperating to form the generally conical shape. The top surfaces 302 preferably transition to each other around the perimeter of the dilator 10 generally smoothly and continuously to minimize or eliminate any damage caused by tearing by jagged edges as the dilator 10 is rotated. The top surface 302 transitions to the ear 308 at or near the proximal end 310 of the top surface 302, and extends to a tip 314 at the distal end 312 of the top surface 302. The tip 314 is configured to assist in forming a leading edge when the dilator 10 is in the closed position to help ease insertion. The top surface 302 also includes threads 316. The threads 316 are configured to allow for improved insertion of the dilator 10 through tissue. For example, a clockwise rotation may urge the dilator 10 distally forward through tissue. By using the threads 316 and turning the dilator 10 as it is inserted, the threads 316 can help advance the dilator 10 through tissue while minimizing the amount of pushing through the tissue that can result in increased damage to tissue. Preferably, the threads 316 are configured generally continuously and smoothly across the blades 300 when they are in the closed position to minimize any jagged edges between the threads of the adjacent blades and any tearing of tissue that may result from such jagged edges.

The side surfaces 304 extend from the top surface 302 generally toward the axis of a cone defined by the top surface 302. Along a proximal portion of the blade 300, the side surfaces 304 extend from the top surface 302 to the bottom surface 306. More distally, in the illustrated embodiment, the side surfaces 304 extend from the top surface toward each other (see FIG. 9.) When the dilator 10 is in the closed position, the side surfaces 304 of adjacent blades 300 may contact to minimize any gap between the blades 300 and also to provide a positive mechanical stop during the closing process.

The bottom surface 306 is generally smooth and defines a portion of generally conical segment. The bottom surface 306 is configured to generally match the slope and shape of the sloped exterior section 133 of the wedge portion 132 of the drive arm 100. When the blades 300 are together in the closed position, the bottom surfaces 306 of the blades 300 cooperate to generally surround the wedge portion 132 of the drive arm 100. The blades 300 are configured so that the top surfaces 302 of the blades cooperate to form a generally cylindrical envelope or volume (with gaps between portions of adjacent blades) when the drive arm is fully advanced distally and the dilator 10 is in the open position.

The ear 308 of the illustrated embodiment is configured to be accepted in the slot 214 of the body 200 and provide for pivotal connection to the body 200, as well as to help provide a positive mechanical stop limiting the movement of the blades 300 and helping to lock the blades 300 in a desired position. The ear 308 includes pin holes 320 that accept pins 400 to allow for pivotal connection to the body 200.

The ear 308 also includes a projection 322 that includes a forward surface 324 and a bottom surface 326. The forward surface 324 is configured to contact the rear surface 134 of the drive arm 100 when the dilator 10 is in the closed position. With the drive arm 100 held in position, the interaction of the forward surface 324 and rear surface 134 acts to lock the blades 300 in place, preventing them from pivoting about the pins 400 to move toward the open position. Further, when the blades 300 are in the open position and the drive arm 100 is actuated proximally, the rear surface 134 of the drive arm 100 contacts and drives against the forward surface 324, thereby pivoting the blade 300 toward the closed position. This provides an example of a first surface on the blade 300 and a first surface on the drive arm 100 that cooperate to provide a drive engagement for closing the blades and a locking engagement for maintaining the blades in a closed position.

The projection 322 also includes a bottom surface 326. The bottom surface 326 is configured to contact the exterior surface 123 of the shaft 122 when the blade 300 is in the open position (i.e., the top surfaces of the blades define a generally cylindrical envelope). Thus, the projection 322 fowls a protrusion that contacts a first surface (the rear surface) to prevent the blade from opening, and a second surface (the exterior surface of the shaft) to limit how far the blade opens.

Operation of one embodiment of the present invention will now be discussed with reference to FIG. 10, which illustrates a perspective view of a dilation and retraction system 500 formed in accordance with an embodiment of the present invention. The system 500 includes a dilator 510 having a drive arm 520, body 530, dilation blades 540, and a handle 550. The handle is removably attached to the drive arm 520. The dilator 510 may be generally similar to the above described dilator 10, and include a threaded exterior. The system 500 also includes a retractor 552 including two retractor blades 560, 570 joined by a retractor frame 580. Additional detail on such retractor blades and a retractor system that may be used in conjunction with the dilator 510 can be found in U.S. patent application Ser. No. 12/959,701 (“Posterior Lumbar Retractor System”, filed Dec. 3, 2010), which is hereby incorporated by reference in its entirety. In FIG. 10, the retractor blades 560, 570 are shown spread apart, but could be drawn closer together to a closed retractor position. While there may be gaps between the retractor blades 560, 570 in the closed position, the retractor blades 560, 570 cooperate to form a generally circular cross-section in the closed position, and may be moved from the closed position to an open position wherein the blades are farther apart and/or angled to each other. The retractor is configured to be slid in the closed position over the dilator 510 when the dilator 10 is in the open position. For example, the dilator 10 may define a generally cylindrical volume having a diameter of about 18 millimeters, and the retractor 552 may define a slightly larger generally circular cross section in its closed position so that the retractor 552 may be slid over the dilator 510 toward the site of interest while minimizing additional tissue damage. In other embodiments, for example, dilators may define volumes having diameter of about 22 millimeters, or 26 millimeters, with the refractor correspondingly sized.

To use the system 500, an incision above the site of interest is made. The dilator 510 is then introduced into the incision and then rotated to use the threaded exterior to help advance the dilator 510 through the tissue and toward the site of interest. For example, the threads of the dilator may be configured so that the dilator may be rotated in a clockwise direction to advance through tissue. To help maintain the dilator in the closed position during the insertion, the threaded engagement of the arm and the body may be configured to prevent distal advance during insertion. For example, if the dilator advances through tissue due to a clockwise rotation, the arm and body may be configured so that the arm advances distally relative to the body when rotated counter-clockwise. Thus, due to the opposite directions of rotation for distal advancement, the arm is not inadvertently advanced during insertion, avoiding the premature opening of the dilator. Further still, in certain embodiments, due to the interaction of the protrusion of the ear of the blade and the rear surface of the arm (discussed above), the clockwise rotation not only avoids inadvertent advancing of the arm, but also urges the arm proximally which helps engage the locking interaction between the awl and the blade, adding further security to the maintenance of the dilator in its closed position during insertion. Put another way, the same direction of rotation that urges the dilator forward through tissue simultaneously urges and/or maintains the dilator in the closed position. Thus, the dilator 510 may be advanced be grasping the handle 550 and turning the handle 550 in the clockwise position while in the closed position.

Once the dilator 510 is in its desired location it may be opened. This may be accomplished without the addition or removal of any parts or additional steps. Instead, the handle 550 is merely rotated in the opposite direction, counter-clockwise in this example. The tapered edge of the dilator 510 in the closed position combined with the threading motion helps reduce tearing and other tissue damage during insertion. The dilator 510 may be grasped or otherwise secured, for example, around the body 530 of the dilator 510 to help ensure that the dilation blades 540 (with their threaded exteriors) do not rotate during this counter-clockwise rotation to open the dilation blades 540. The handle 550 is rotated as described, urging the drive arm 520 distally and the dilation blades 540 apart, until the dilator 510 has reached its open position (the dilator 510 is shown in FIG. 10 in the open position, with the distal end of the dilator defining a generally cylindrical volume or envelope). This opening motion can help to spread tissue instead of tearing tissue which may be caused by the insertion of blunt dilators.

The retractor 552 may now be inserted over the dilator 510. The handle 540 is configured to be removable in the illustrated embodiment to ease insertion of the retractor 550. Once the retractor 552 is inserted, the retractor blades 560, 570 may be moved to their open position and secured in place, the drive arm 520 rotated to return the dilator 510 to the closed position, and the dilator 510 removed in one piece, in contrast to certain known procedures using multiple dilators that must be inserted and/or removed one at a time in a number of steps. In other embodiments, individual retractor blades may be introduced and positioned as desired, or other types of retractors advanced to the site along the dilator and opened and/or positioned as desired. Thus, quick, efficient, convenient dilation and retraction is provided in a way that reduces tissue damage caused, for example, by multiple blunt dilation members being inserted over each other. At the same time, the system 500 allows for a low number of steps along with the flexibility and enlarged access area made possible by the use of the retractor blades. Use of a separate, expandable retractor and/or retractor blades that slide over the dilator allows for a larger and more adjustable access area than would be possible if just using the cylindrical volume defined by the interior of the blades of the dilator.

For example, use of certain known dilation systems includes the insertion of a K wire and an X-Ray to confirm the correct position. Then, progressively larger dilators in steps (for example, from 2 to 5 steps may be used) are introduced, with a retractor introduced after the last dilator. This procedure, however, requires the use of a K wire, which may be inadvertently advanced into the spinal cord, which may result in injury including potential paralysis. The stepped dilation also increases operating time.

In contrast, use of certain embodiments of the present invention includes twisting the dilator into the incision, attaching the dilator to a table mount to secure in position, and performing an X-Ray to confirm the correct position. Then, the distal end of the dilator is opened and the retractor introduced. Thus, by eliminating the K wire, safety may be improved. Elimination of the stepped dilation reduced operating time. Use of a table mount helps maintain the dilator in the correct position. For example, a table mount could attach to a nipple at the top of the handle, or, as another example, a table mount could be secured to the drive arm or the body of the dilator.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present surgical port, which is set forth in the following claims. In addition, it should be understood that aspects of the various embodiments may be interchanged both in whole or in part. Therefore, the spirit and scope of the appended claims should not be limited to the description of the preferred versions contained therein.

What is claimed is:

1. A dilator for providing access to a surgical site of interest, the dilator including:
a body including a cavity;
an arm configured to be accepted by the cavity of the body, the arm including a distal portion and a proximal portion; and
a plurality of articulating dilation members pivotally connected to the body and operably connected to the distal portion of the arm, wherein the articulating dilation members are movable between a closed position and an open position, wherein the articulating dilation members are moved from the closed position toward the open position by the arm when the arm is articulated in a first linear direction, and moved from the open position to the closed position by the arm when the arm is articulated in a second linear direction that is opposite to the first linear direction, wherein the arm and the plurality of articulating dilation members include cooperating surfaces that maintain the articulating dilation members in the closed position when the arm is held in place during insertion of the dilator.

2. The dilator of claim 1 wherein the cavity of the body includes a threaded section and the arm includes an intermediate portion including a threaded portion, wherein the threaded portion of the body accepts the threaded portion of the arm wherein the arm may be articulated linearly relative to the body when the arm is rotated.

3. The dilator of claim 2 wherein the distal portion of the arm includes a conically shaped portion including a sloped surface having a distally reduced cross sectional area, wherein the sloped surface actuates the articulating dilation members toward the open position when the arm is articulated distally.

4. The dilator of claim 3 wherein the distal portion of the arm includes a rear surface located proximally of the sloped surface and each articulating dilation member includes an ear, wherein the rear surface of the arm contacts the ears and moves the articulating dilation members from the open position to the closed position when the arm is articulated proximally.

5. The dilator of claim 1 wherein the distal portion of the arm includes a rear surface and each articulating dilation member includes an ear, wherein the rear surface of the arm contacts the ears and prevents the articulating dilation members from pivoting toward the open position when the articulating dilation members are in the closed position.

6. The dilator of claim 1 wherein the articulating dilation members comprise external surfaces, the external surfaces comprising threads, wherein the articulating dilation members define a substantially continuous threaded surface when the articulating dilation members are in the closed position.

7. The dilator of claim 6, wherein the cavity of the body includes a threaded section and the arm includes an intermediate portion including a threaded portion, wherein the threaded portion of the body accepts the threaded portion of the arm wherein the arm may be articulated linearly relative to the body when rotated, and wherein rotation in a first rotational direction advances the arm in the first linear direction relative to the body to move the dilator toward the open position and rotation in a direction opposite to the first rotational direction advances the articulating dilation members through tissue.

8. The dilator of claim 1, wherein at least one of the articulating dilation members comprises an ear having a protrusion, wherein the protrusion contacts a first surface of the arm to prevent opening and contacts a second surface of the arm to limit opening.

9. A dilation and retraction system for providing access to a surgical site of interest, the dilator including:
a dilator, the dilator including:
a body including a cavity;
an arm configured to be accepted by the cavity of the body, the arm including a distal portion and a proximal portion; and
a plurality of articulating dilation members pivotally connected to the body and operably connected to the distal portion of the arm, wherein the articulating dilation members are movable between a closed position and an open position, wherein the articulating dilation members are moved from the closed position toward the open position by the arm when the arm is articulated distally, and moved from the open position to the closed position by the arm when the arm is articulated proximally, wherein the arm and the plurality of articulating dilation members include cooperating surfaces that maintain the articulating dilation members in the closed position when the arm is held in place during insertion of the dilator; and
a retractor configured to be slid over the dilator when the dilator is in the open position.

10. The dilation and retraction system of claim 9 wherein the retractor comprises a plurality of retractor blades that are configured to be slid over the dilator in a closed retractor position and moved to a open retractor position.

11. The dilation and retraction system of claim 9 wherein the dilator defines a generally cylindrical envelope when in the open position, and the retractor defines a generally circular cross-section configured to be slid over the dilator.

12. The dilation and retraction system of claim 9 wherein the cavity of the body includes a threaded section and the arm includes an intermediate portion including a threaded portion, wherein the threaded portion of the arm accepts the threaded portion of the arm wherein the arm may be articulated linearly relative to the body when rotated.

13. The dilation and retraction system of claim 12 wherein the distal portion of the arm includes a conically shaped portion including a sloped surface having a distally reduced cross sectional area, wherein the sloped surface actuates the articulating dilation members toward the open position when the arm is articulated distally.

14. The dilation and retraction system of claim 9 wherein the articulating dilation members comprise external surfaces, the external surfaces comprising threads, wherein the articulating dilation members define a substantially continuous threaded surface when the articulating dilation members are in the closed position.

15. The dilation and retraction system of claim 14 wherein the cavity of the body includes a threaded section and the arm includes an intermediate portion including a threaded portion, wherein the threaded portion of the body accepts the threaded portion of the arm wherein the arm may be articulated linearly relative to the body when the arm is rotated, and wherein rotation in a first direction advances the arm distally relative to the body and rotation in a direction opposite to the first direction advances the articulating dilation members through tissue.

16. The dilation and retraction system of claim 9 wherein at least one of the articulating dilation members comprises an ear having a protrusion, wherein the protrusion contacts a first surface of the arm to prevent opening and contacts a second surface of the arm to limit opening.

17. A method of providing access to a surgical site, the method including:
making an incision;
advancing a dilator having articulating dilation members in a closed position into a patient toward a site of interest through the incision, the dilator having a generally conical distal end when in the closed position;
moving the articulating dilation members to an open position wherein the distal end of the dilator defines a generally cylindrical volume in the open position;
inserting a retractor over the dilator toward the site of interest;
positioning the retractor; and
removing the dilator to provide access to the site of interest.

18. The method of claim 17 wherein the dilator includes a threaded arm that spreads the articulating dilation members when the threaded arm is advanced distally, and wherein moving the articulating dilation members to the open position includes rotating the threaded arm in a direction to advance the threaded arm distally.

19. The method of claim 17 wherein the dilator includes a threaded exterior, wherein advancing the dilator includes rotating the dilator in a direction to advance the dilator distally through tissue.

20. The method of claim 17 wherein the dilator includes a threaded arm that spreads the articulated dilation members when the threaded arm is advanced distally, and wherein the dilator includes a threaded exterior, wherein advancing the dilator includes rotating the dilator in a first direction, and wherein moving the articulating dilation members to the open position includes rotating the threaded arm in a second direction, the second direction being opposite to the first direction.
